(19) United States
(12) Patent Application Publication
(10) Pub. No.: US 2017/0349881 A1
(43) Pub. Date: Dec. 7, 2017

(54) TRAIT INDUCTION METHOD OF UNDIFFERENTIATED CELLS

(71) Applicant: Tokyo Ohka Kogyo Co., Ltd., Kanagawa (JP)

(72) Inventors: Emi MAENO, Kawasaki-shi (JP); Yasuhiko TABATA, Kyoto (JP)

(73) Assignee: Tokyo Ohka Kogyo Co., Ltd., Kanagawa (JP)

(21) Appl. No.: 15/610,731

(22) Filed: Jun. 1, 2017

(30) Foreign Application Priority Data
Jun. 2, 2016 (JP) 2016-110923

(51) Int. Cl. C12N 5/0786

(52) U.S. Cl. CPC C12N 5/0645; C12N 2535/10; C12N 2533/30; C12N 2501/2304; C12N 2501/90; C12N 2501/24; C12N 2506/03

(57) ABSTRACT

A trait induction method of undifferentiated cells is provided, including: culturing undifferentiated cells on abase material which has an uneven pattern on the surface to which the cells adhere and of which the width of the unevenness is 1 nm to 1,000 nm.

### BACKGROUND OF THE INVENTION

### Field of the Invention

The present invention relates to a trait induction method of undifferentiated cells.

Priority is claimed on Japanese Patent Application No. 2016-110923, filed on Jun. 2, 2016, the content of which is incorporated herein by reference.

### Description of Related Art

In regenerative medicine, it is necessary to control differentiation of cells, and it is known that, if a differentiation mechanism of cells is destroyed, this causes a neoplastic disease or the like. For this reason, a technique for controlling differentiation in which undifferentiated cells are efficiently induced in a constant direction is required. As the method of inducing differentiation of cells, for example, a method of using support cells, genes, or the like or a method of using electrical stimulation has been developed.

Examples of the method of using support cells, genes, or the like include a method of inducing differentiation of ES cells into endodermal cells using cells derived from the mesoderm as support cells (for example, refer to Republished Japanese Translation No. WO2006/126574 of the PCT International Publication for patent applications).

In addition, examples of the method of using electrical stimulation include a method of inducing differentiation of cells by repeatedly applying an electric pulse or an impulse into undifferentiated cells at a predetermined cycle (for example, refer to Japanese Unexamined Patent Application, First Publication No. 2004-105148).

### SUMMARY OF THE INVENTION

In the method of using support cells, genes, or the like, it is necessary to remove the used support cells or the genes after the undifferentiated cells are differentiated into desired cells. In addition, undifferentiated cells remain since it is impossible to induce differentiation of all of the undifferentiated cells. Therefore, there is a problem in that these undifferentiated cells become cancerous.

Furthermore, there is another problem in that differentiated cells become cancerous due to the influence of support cells or genes to be used.

In the method of using electrical stimulation, it is unnecessary to remove residual substances such as support cells, genes, or the like. However, the problem in which residual undifferentiated cells become cancerous has not yet been solved.

The present invention has been made in consideration of the above-described circumstances, and an object of the present invention is to provide a method of simply and efficiently inducing undifferentiated cells to have a constant trait.

The present inventors have paid attention to the fact that the trait induction of undifferentiated cells is caused by stimulation due to nanoscale molecules on the cell surface, and have completed the present invention.

That is, the present invention includes the following embodiment.

According to the embodiment of the present invention, a trait induction method of undifferentiated cells is provided, including: culturing undifferentiated cells on a base material which has an uneven pattern on the surface to which the cells adhere and of which the width of the unevenness is 1 nm to 1,000 nm.

According to the present invention, it is possible to simply and efficiently induce undifferentiated cells to have a constant trait.

### BRIEF DESCRIPTION OF THE DRAWINGS

FIG. 1A shows an enlarged plan view and an enlarged front view of a base material (pattern: linear shape) used in a trait induction method of the present embodiment. FIG. 1B shows an enlarged plan view and an enlarged front view of a base material (pattern: dot shape) used in the trait induction method of the present embodiment.

FIGS. 2A to 2D are graphs showing quantitative results of the production amount of nitrogen monoxide (NO) of RAW264 cells in Example 1.

FIGS. 3A to 3D are graphs showing measurement results of arginase activity of RAW264 cells in Example 1.

FIGS. 4A and 4B are graphs showing measurement results of acetylcholine esterase (ACNE) activity of PC12 cells in Example 2.

FIGS. 5A and 5B are graphs showing quantitative results of the production amount of dopamine of PC12 cells in Example 2.

### DETAILED DESCRIPTION OF THE INVENTION

### Trait Induction Method of Undifferentiated Cells

In one embodiment, the present invention provides a trait induction method of undifferentiated cells, including: culturing undifferentiated cells on a base material which has an uneven pattern on the surface to which the cells adhere and of which the width of the unevenness is 1 nm to 1,000 nm.

In the trait induction method of the present embodiment, it is possible to simply and efficiently induce undifferentiated cells in a constant differentiation direction through stimulation applied on the surfaces of the undifferentiated cells by culturing the undifferentiated cells using a base material having an uneven nano pattern on the surface to which cells adhere. Furthermore, in the cells of which differentiation is induced, it is possible to perform trait induction of cells by performing culture using a base material having an uneven nano pattern on the surface to which the cells adhere. For example, it is possible to perform trait induction of macrophages to inflammatory type (M1) macrophages, anti-inflammatory type (M2) macrophages, or the like through the trait induction method of the present embodiment. In other words, the method of the present embodiment is a method of controlling the proliferation, differentiation and transformation of undifferentiated cells. Accordingly, in the present specification, the “trait induction” includes “differentiation induction”.

In the present specification, the “undifferentiated cells” are not particularly limited as long as they are cells having a differentiation ability. Examples of the undifferentiated cells include stem cells or precursor cells.

The term stem cells refers to cells having both an ability to duplicate themselves and an ability to be differentiated into other cells with a plurality of systems. Examples of the stem cells include pluripotent cells such as embryonic stem cells (ES cells), embryonic tumor cells, embryonic germ stem cells (EG cells), and artificial pluripotent stem cells (iPS cells); somatic stem cells such as mesenchymal stem cells (MSC), neural stem cells, hematopoietic stem cells, vascular stem cells, amniotic cells, cord blood cells, bone marrow-derived cells, myocardial stem cells, adipose-derived stem cells, hepatic stem cells, pancreatic stem cells, muscle stem cells, germ stem cells, intestinal stem cells, cancer stem cells, and hair follicle stem cells.

The term precursor cells refers to cells in a state in the middle of differentiation from stem cells into specific somatic cells or germ cells. Examples of the precursor cells include precursor cells in the middle of being differentiated into cells constituting tissue of the skin, the kidneys, the spleen, the adrenal gland, the liver, the lung, the ovary, the pancreas, the uterus, the stomach, the small intestine, the large intestine, the bladder, the prostate, the testis, the thymus, muscles, connective tissue, bone, cartilage, vascular tissue, blood, the heart, the eyes, the brain, nerves, and the like.

Substrate

In the trait induction method of the present embodiment, a substrate to be used has a pattern of unevenness on the base plate. In addition, the substrate is not particularly limited as long as the substrate is not deformed when culturing cells or by pretreatment such as sterilization treatment. The whole of the substrate may be composed of the same material, and the substrate may be composed of a pattern of unevenness made of different materials, and a base plate for supporting the pattern of unevenness.

Examples of the form of the substrate include a multi-well plate or a Petri dish in which an arbitrary number of wells are disposed. Examples of the number of wells include 6, 12, 24, 96, 384, and 1,536 per plate.

Base Plate

In a case where the substrate may be composed of the pattern of unevenness made of different materials, and the base plate for supporting the pattern of unevenness, the material of the base plate is not particularly limited as long as the base plate is used for cell culture applications. More specific examples of the material of the base plate include glass, polyethylene terephthalate, polycarbonate, cycloolefin polymer, polydimethylsiloxane, polystyrene, and the like. By using these materials, autofluorescent materials can be reduced, and the cultured cells can be observed with a fluorescence microscope.

Uneven Pattern

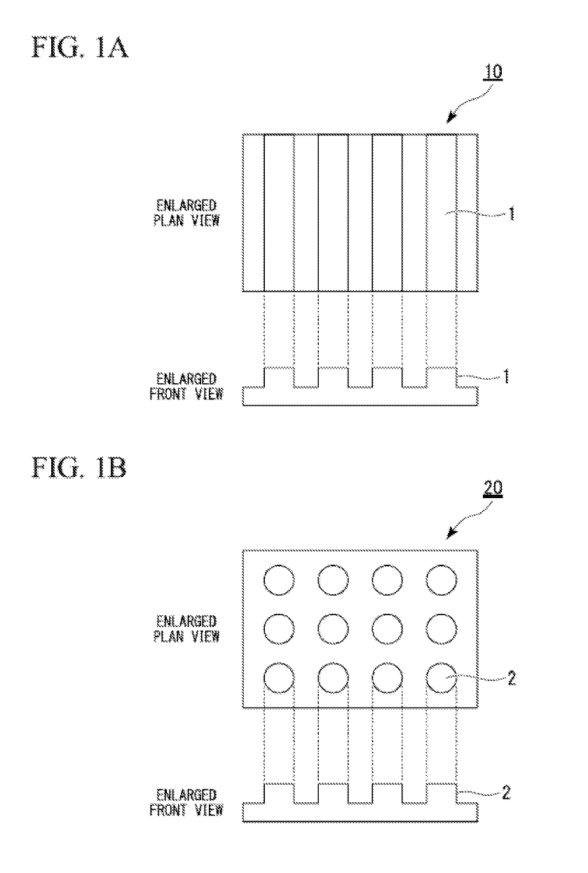

Examples of patterns of unevenness include a lattice shape, a radial shape, a polygon continuous shape on a flat surface (for example, a honeycomb structure or the like), a labyrinthine shape, a line shape, a dot shape, or the like. FIGS. 1A and 1B are an enlarged plan view and an enlarged front view of the cell culture base member for trait induction control of a macrophage according to the embodiment. FIG. 1A illustrates a case where the pattern of unevenness is a line shape, and FIG. 1B illustrates a case where the pattern of unevenness is a dot shape. In the enlarged front view of FIG. 1A, the shape of a projection portion is a rectangular parallelepiped line shape, but it is not limited thereto, and the shape of the projection portion may be a rectangular column shape (including a rectangular parallelepiped and a cube in a rectangular column), a truncated pyramidal shape, a semicircular column shape (including a semi-elliptical column in a semicircular column), a truncated conical shape (including an elliptical frustum and a biconical truncated cone in a truncated cone), or the like.

In addition, FIG. 1B illustrates a dot shape in which the transverse section of the projection portion is a circular shape and the vertical section of the projection portion is a rectangular shape (that is, the shape of the projection portion is circular column shape), but it is not limited thereto, and the transverse section or the vertical section of the projection portion may be a polygonal shape such as triangle and square, a circular shape (including a substantially circular shape, an elliptical shape, a substantially elliptical shape, a semicircular shape, and a fan shape in a circular shape), a trapezoidal shape, a wave shape, or the like. That is, examples of the shape of the projection portion include a rectangular column shape (including a rectangular parallelepiped and a cube in a rectangular column), a truncated pyramidal shape (including a truncated bipyramid in a truncated pyramid), a circular column shape (including an elliptic column, a semicircular column, a semi-elliptical column, and a sectoral column in a circular column), and a truncated conical shape (including an elliptical frustum and a biconical truncated cone in a truncated cone), or the like, but it is not limited thereto.

The width of the convave portion and the projection portion is preferably 1 nm or more and 1,000 nm or less, more preferably 10 nm or more and 1,000 nm or less, and further preferably 100 nm or more and 1,000 nm or less. When the width is within the above range, it is possible to stimulate the cell surface of the undifferentiated cells and to simply and efficiently induce undifferentiated cells in a constant differentiation direction. In a case where the shape of the projection portion is a circular column shape or a truncated conical shape, the width represents the diameter of the upper surface of the projection portion.

The distance to the surface of the projection portion of the uneven pattern is preferably 10 nm to 100 μm. In a case where the distance to the surface of the projection portion is within the above range, autofluorescence of the substrate is easily suppressed. Therefore, when the substrate having the distance within the above range is used, it is easy to observe the cultured cells by the fluorescence microscope.

Method of Forming Uneven Pattern

A method of forming the uneven pattern is not particularly limited. Examples of the method of forming the uneven pattern include a photolithography method in which a photosensitive composition layer formed on a surface of a substrate for supporting an uneven pattern is selectively exposed, and thereafter a portion corresponding to a convave portion is removed from the exposed photosensitive composition layer with a developing solution, an imprinting method of curing an imprint material after pressing a pressing mold having a pattern of unevenness on a layer of the imprint material formed on a base plate surface, a method in which a mask for covering a portion corresponding to a projection portion is provided on a base plate surface, and thereafter a convave portion is formed on the base plate surface by a chemical treatment such as etching, a method of grinding a base plate surface by sand blasting or various machine tools, a method of attaching a material constituting a projection portion of a pattern having a predetermined shape to a base plate surface, and the like. For the photolithography method and the imprinting method, a photosensitive resin composition used for various purposes in the related art and a photosensitive spin-on-glass (SOG) material can be used without particular limitation.

Photosensitive Resin Composition

Examples of the photosensitive resin composition used for forming the uneven pattern include a photosensitive resin composition containing a resin component, a cationic polymerization initiator, and a solvent, and the like. The photosensitive resin composition may be any of a positive type and a negative type.

The resin component is not particularly limited as long as the resin component can be used for cell culture, for example. Among these, a polymer of a compound having an ethylenic unsaturated bond is preferable. Examples of the polymerizable functional group contained in the compound having an ethylenic unsaturated bond include a (meth)acryloyl group, a vinyl group, an allyl group, and the like. As the compound having the ethylenic unsaturated bond, for example, a monofunctional, a difunctional, or a trifunctional or higher polyfunctional, (meth)acrylate compound, (meth)acrylamide compound, vinyl compound, allyl compound, or the like can be used. These compounds having the ethylenic unsaturated bond can be used alone or in a combination of two or more.

Examples of the polyfunctional compound having the ethylenic unsaturated bond include trifunctional or higher acrylates such as trimethylolpropane tri(meth)acrylate, pentaerythritol tetra(meth)acrylate, ethylene oxide modified pentaerythritol tetra(meth)acrylate, propylene oxide modified pentaerythritol tetra(meth)acrylate, dipentaerythritol penta(meth)acrylate, dipentaerythritol hexa(meth)acrylate, and the like; a polyfunctional urethane (meth)acrylate obtained by reacting a polyisocyanate compound and a hydroxy group-containing (meth)acrylate monomer; and a condensate of polyhydric alcohol and N-methylol(meth)acrylamide, and the like. These polyfunctional compounds can be used alone or in a combination of two or more.

Examples of the di functional compound having the ethylenic unsaturated bond include polyethylene glycol di(meth)acrylate, polypropylene glycol di(meth)acrylate, polyethylene polypropylene glycol di(meth)acrylate, ethylene glycol di(meth)acrylate, propylene glycol di(meth)acrylate, polyethylene poly trimethylolpropane di(meth)acrylate, 2-(meth)acryloyloxy-2-hydroxypropyl phthalate, 2-(meth)acryloyloxyethyl-2-hydroxyethyl phthalate, a compound obtained by reacting a glycidyl group-containing compound with α,β-unsaturated carboxylic acid, urethane monomers, γ-chloro-β-hydroxypropyl-β′-(meth)acryloyloxyethyl-o-phthalate, β-hydroxyethyl-β′-(meth)acryloyloxyethyl-o-phthalate, β-hydroxypropyl-β′-(meth)acryloyloxyethyl-o-phthalate, and the like.

Examples of the compound obtained by reacting the glycidyl group-containing compound with α,β-unsaturated carboxylic acid include triglycerol di(meth)acrylate, and the like. Examples of the urethane monomer include addition reaction products of a (meth)acrylic monomer having a hydroxyl group at the β position with isophorone diisocyanate, 2,6-toluene diisocyanate, 2,4-toluene diisocyanate, 1,6-hexamethylene diisocyanate, or the like, EO modified urethane di(meth)acrylate, EO, PO modified urethane di(meth)acrylate, and the like.

Examples of monofunctional compounds having the ethylenic unsaturated bond include (meth)acrylic acid esters, (meth)acrylamides, allyl compounds, vinyl ethers, vinyl esters, styrenes, and the like. These compounds can be used alone or in a combination of two or more.

Examples of the (meth)acrylate esters include methyl (meth)acrylate, ethyl (meth)acrylate, propyl (meth)acrylate, amyl (meth)acrylate, t-octyl(meth)acrylate, chloroethyl (meth)acrylate, 2,2-dimethylhydroxypropyl (meth)acrylate, 2-hydroxyethyl (meth)acrylate, trimethylolpropane mono(meth)acrylate, benzyl (meth)acrylate, furfuryl (meth)acrylate, phenyl (meth)acrylate, (meth)acrylate of EO adduct of phenol, (meth)acrylate of PO adduct of phenol, (meth)acrylate of EO/PO co-adduct of phenol, ethylene glycol mono(meth)acrylate, diethylene glycol mono(meth)acrylate, triethylene glycol mono(meth)acrylate, polyethylene glycol mono(meth)acrylate, 2-methoxyethyl (meth)acrylate, diethylene glycol monomethyl ether mono(meth)acrylate, triethylene glycol monomethyl ether mono(meth)acrylate, polyethylene glycol monoethyl ether mono(meth)acrylate, propylene glycol mono(meth)acrylate, dipropylene glycol mono(meth)acrylate, tripropylene glycol mono(meth)acrylate, polypropylene glycol mono(meth)acrylate, propylene glycol monomethyl ether mono(meth)acrylate, dipropylene glycol monomethyl ether mono(meth)acrylate, tripropylene glycol monomethyl ether mono(meth)acrylate, polypropylene glycol monomethyl ether mono(meth)acrylate, mono(meth)acrylate of EO/PO copolymer, monomethyl ether mono(meth)acrylate of EO/PO copolymer, and the like.

Examples of the (meth)acrylamides include (meth)acrylamide, N-alkyl (meth)acrylamide, N-allyl (meth)acrylamide, N,N-dialkyl (meth)acrylamide, N,N-allyl (meth)acrylamide, N-methyl-N-phenyl (meth)acrylamide, N-hydroxyethyl-N-methyl (meth)acrylamide, and the like.

Examples of the vinyl ethers include alkyl vinyl ethers such as hexyl vinyl ether, octyl vinyl ether, decyl vinyl ether, ethylhexyl vinyl ether, methoxyethyl vinyl ether, ethoxyethyl vinyl ether, chloroethyl vinyl ether, 1-methyl-2,2-dimethylpropyl vinyl ether, 2-ethyl butyl vinyl ether, hydroxyethyl vinyl ether, diethylene glycol vinyl ether, dimethylamino ethyl vinyl ether, diethyl aminoethyl vinyl ether, butyl aminoethyl vinyl ether, benzyl vinyl ether, tetrahydrofurfuryl vinyl ether, and the like; vinyl allyl ethers such as vinyl phenyl ether, vinyl tolyl ether, vinyl chlorophenyl ether, vinyl-2,4-dichlorphenyl ether, vinyl naphthyl ether, vinyl anthranyl ether, and the like.

Examples of the vinyl esters include vinyl butyrate, vinyl isobutyrate, vinyl trimethyl acetate, vinyl diethyl acetate, vinyl valate, vinyl caproate, vinyl chloroacetate, vinyl dichloroacetate, vinyl methoxy acetate, vinyl butoxy acetate, vinyl phenylacetate, vinyl acetoacetate, vinyl lactate, vinyl-β-phenylbutyrate, vinyl benzoate, vinyl salicylate, vinyl chlorobenzoate, vinyl tetrachlorobenzoate, vinyl naphthoate, and the like.

Examples of the styrenes include styrene; alkyl styrene such as methyl styrene, dimethyl styrene, trimethylstyrene, ethylstyrene, diethylstyrene, isopropylstyrene, butylstyrene, hexylstyrene, cyclohexylstyrene, decylstyrene, benzylstyrene, chloromethylstyrene, trifluoromethylstyrene, ethoxymethylstyrene, acetoxymethylstyrene, and the like; alkoxystyrene such as methoxystyrene, 4-methoxy-3-methylstyrene, dimethoxystyrene, and the like; halostyrene such as chlorostyrene, dichlorostyrene, trichlorostyrene, tetrachlorostyrene, pentachlorostyrene, bromostyrene, dibromostyrene, iodostyrene, fluorostyrene, trifluorostyrene, 2-bromo-4-trifluoromethylstyrene, 4-fluoro-3-trifluoromethylstyrene, and the like.

The cationic polymerization initiator is one that generates a cation upon irradiation with radiation such as ultraviolet ray, far ultraviolet ray, an excimer laser such as KrF, ArF or the like, X-ray and electron beam, and the like, and the cation thereof is a compound that can be a polymerization initiator.

As the cationic polymerization initiator, for example, an onium salt type cationic polymerization initiator such as an iodonium salt or a sulfonium salt can be used. The anion to be a counter ion of the onium ion constituting the onium salt type cationic polymerization initiator is preferably a fluorinated alkyl fluorophosphate anion, a hexafluorophosphate anion, or a hexafluoroantimonate acid anion (SbF₆⁻).

The solvent contained in the photosensitive resin composition is not particularly limited as long as the solvent can prepare a uniform photosensitive resin composition and does not hinder the effect of exposure. The boiling point of the solvent is preferably 50° C. to 200° C.

Specific examples of the solvent include aliphatic hydrocarbons such as hexane, heptane, octane, decane, and cyclohexane; alcohols such as methanol, ethanol, 1-propanol, 2-propanol, and 1-butanol; acetone, methyl ethyl ketone, methyl isobutyl ketone, 2-heptanone, ethyl lactate, propylene glycol monomethyl ether acetate, propylene glycol monomethyl ether, ethyl acetate, butyl acetate, ethylene glycol dimethyl ether, propylene glycol dimethyl ether, methyl cellosolve, ethyl cellosolve, dibutyl ether, methyl-3-methoxypropionate, propylene glycol mono propyl ether, butyl cellosolve, diethylene glycol diethyl ether, hexylene glycol, cyclohexanone, propylene glycol monoethyl ether, ethyl pyruvate, ethyl cellosolve acetate, and the like. These may be used alone or in a combination of two or more.

In addition to the resin component, the cationic polymerization initiator, and the solvent, the photosensitive resin composition may contain various additives used in the photosensitive resin composition in the related art. Examples of such additives include additional resins, sensitizers, plasticizers, stabilizers, colorants, coupling agents, leveling agents, and the like.

The photosensitive resin composition can be prepared by mixing (dispersing and kneading) each of the above components with a stirrer such as a triple roll mill, a ball mill, a sand mill, or the like and filtering with a filter such as a 5 □m membrane filter if required.

Method of Preparing Substrate

The method of preparing the substrate is not particularly limited as long as it is a method capable of forming the substrate having a desired pattern by exposing and curing the above-described photosensitive resin composition. As a method of preparing a cell culture substrate, for example, a method including a coating process of coating a photosensitive resin composition onto a base plate to form a coating film and an exposure process of exposing the coating film on the base plate to cure the coating film can be mentioned. The method of preparing the substrate may include a detachment process of detaching the exposed coating film from the base plate after curing the coating film on the base plate by exposure if required.

In the coating process, the base plate on which the photosensitive resin composition is coated is not particularly limited as long as the base plate does not cause deformation or deterioration in the process of preparing the substrate. As the material of the base plate, the same base plate as the above “Base plate” can be mentioned.

As a method of forming a pattern of unevenness, the same method as the above “Method of Forming Uneven Pattern” can be mentioned.

The method of forming the coating film on the base plate is not particularly limited, and examples thereof include a method in which a predetermined amount of the photosensitive resin composition is dropped onto the base plate, a method of using a contact transfer type coating applicator such as a roll coater, a reverse coater, a bar coater, or the like, and a method of using a non-contact type coating applicator such as a spinner (rotary coating applicator), a curtain flow coater, or the like.

After the coating film is formed, the base plate provided with the coating film may be placed under a reduced pressure condition to degas the coating film.

In the exposure process, the method of exposing the coating film is not particularly limited as long as the coating film can be satisfactorily cured. For the exposure, for example, a light source emitting ultraviolet rays such as a high-pressure mercury vapor lamp, an ultrahigh pressure mercury vapor lamp, a xenon lamp, a carbon arc lamp or the like may be used. The exposure amount at the time of exposing the coating film is appropriately determined in consideration of the composition of the photosensitive resin composition, the film thickness of the coating film, and the like. Typically, the exposure amount when the coating film is exposed is preferably 10 to 100,000 mJ/cm², and more preferably 100 to 50,000 mJ/cm².

The method of exposing the coating film is not particularly limited, but the coating film may be first exposed to the atmosphere to partially cure the coating film. In this manner, in the exposure process, it is possible to prevent the photosensitive resin composition from protruding from the base plate, and thereafter to expose the coating film in water. If the coating film is exposed in water without exposure to the atmosphere, the coating film may dissolve in water in some cases. When the coating film is exposed to the atmosphere and thereafter the coating film is exposed in water, radical polymerization inhibition due to oxygen can be reduced and a good cured film can be obtained.

In addition, the exposure process may include exposure of the coating film in a vacuum. When the coating film is exposed to the vacuum, the coating film of the photosensitive resin composition can be cured in a state of being in close contact with the base plate, and a substrate having a desired pattern is easily formed. In addition, in a case where the coating film is exposed to the vacuum, exposure may be performed while applying pressure to the coating film from the upper surface of the base plate. In this case, the coating film of the photosensitive resin composition can be cured in a state of being in close contact with the base plate. When the exposure process includes exposure to the vacuum or exposure to the vacuum while applying pressure, specifically, in a case where a substrate is formed by using a mold corresponding to the pattern of unevenness provided in the substrate, it is possible to accurately transfer the uneven pattern of the mold to the substrate. By exposing the coating film under such conditions, shrinkage upon curing of the photosensitive resin composition is suppressed, so that the uneven pattern of the mold can be accurately transferred to the substrate.

As a method of exposing the coating film to the vacuum, for example, a method in which the surface of the coating film is coated with a film such as a PET film, and thereafter the coating film is exposed at least in a state where a space between the film and the coating film is vacuumed can be mentioned. In a case of exposing while applying pressure to the coating film, as a method of applying pressure to the coating film, for example, a method such as negative pressure exposure can be mentioned.

The coating film that is exposed and cured by the method as described above is used as the substrate after detaching from the mold if required.

In addition, the exposed and cured coating film may be subjected to a plasma treatment. By subjecting the cured coating film to the plasma treatment, it is possible to form the substrate to which the cell is likely to adhere. Plasma used for the plasma treatment is not particularly limited, but examples thereof include O₂ plasma, N₂ plasma, CF₄ plasma, and the like. The timing of the plasma treatment is not particularly limited, and the plasma treatment may be performed at any timing before or after detaching the cured coating film from the base plate.

Furthermore, the substrate detached from the mold may be rinsed with a rinsing liquid. When the substrate is rinsed with the rinsing liquid, a compound which can cause cytotoxicity such as an unreacted photopolymerizable monomer or photopolymerization initiator can be removed from the surface of the substrate. Examples of the rinsing liquid include organic solvents such as propylene glycol-1-methyl ether acetate (PGMEA), isopropyl alcohol (IPA), and acetone, water, and the like.

Culture Step

In the trait induction method of the present invention, undifferentiated cells are cultured using the above-described base material. The temperature for culture is preferably 25° C. to 40° C. The culture period of time can be appropriately set in accordance with the types of cells, and is preferably 1 hour to 100 hours. Proliferation and trait induction of undifferentiated cells are promoted using the above-described base material, and it is possible to differentiate undifferentiated cells in a short period of time through a conventional method.

Examples of the undifferentiated cells to be used include stem cells or precursor cells. More specific examples of the stem cells and the precursor cells include the same cells as those in the above-described “Trait Induction Method of Undifferentiated Cells”.

A medium to be used may be a basic medium containing components (such as inorganic salts, carbohydrates, hormones, essential amino acids, nonessential amino acids, and vitamins) or the like required for survival and proliferation of cells, and can be appropriately selected in accordance with the types of cells. Examples thereof include Dulbecco's Modified Eagle's Medium (DMEM), Minimum Essential Medium (MEM), RPMI-1640, Basal Medium Eagle (BME), Dulbecco s Modified Eagle's Medium: Nutrient Mixture F-12 (DMEM/F-12), and Glasgow Minimum Essential Medium (Glasgow MEM). In the culture step, a medium may be appropriately replaced with a new medium in accordance with the proliferation rate of cells.

In addition, compounds for inducing differentiation of undifferentiated cells may be added to a medium to be used. By adding the compound thereto, it is possible to further increase the speed of the differentiation and to control the differentiation in a constant differentiation direction. The compounds for inducing differentiation of undifferentiated cells can be appropriately selected in accordance with the type of cells to be used.

Application

In addition, in the trait induction method of the present embodiment, it is possible to obtain a medical material of which biocompatibility is improved, by processing the above-described base material onto the surface of the medical material. Furthermore, with the use of the medical material, it is possible to control the differentiation of undifferentiated cells and to effectively regenerate tissue.

Examples of the medical material include medical molded bodies for a scaffold to be used for tissue regeneration or transplantation tissue formation of nerves, the heart, blood vessels, cartilage, the skin, the cornea, the kidneys, the liver, hair, cardiac muscles, muscles, tendon, or the like; medical molded bodies such as an aneurysm coil, an embolic substance, an artificial nerve, an artificial mucous membrane, an artificial esophagus, an artificial trachea, an artificial blood vessel, an artificial valve, an artificial thoracic wall, an artificial pericardium, an artificial cardiac muscle, an artificial diaphragm, an artificial peritoneum, an artificial ligament, an artificial tendon, an artificial cornea, an artificial skin, an artificial joint, an artificial cartilage, a dental material, and an intraocular lens, for implanting in a living body; surgical suture, surgical filling material, surgical reinforcement material, wound protection material, bone fracture bonding material, a catheter, a syringe, an infusion or blood bag, a blood filter, and a material for extracorporeal circulation, but is not limited thereto.

### EXAMPLES

Hereinafter, the present invention will be described with reference to Examples, but the present invention is not limited to the following Examples.

### Preparation Example 1

### Preparation of Substrate by Directed Self-Assembly (DSA)

(1) 0.2 mL of a propylene glycol monomethyl ether acetate solution containing 2 wt % of a block copolymer (number average molecular weight 18,000-b-18,000) of polystyrene and polymethyl methacrylate was dropped on a smooth surface of one sheet of a 0.8 cm×0.8 cm glass base plate (manufactured by Hiraoka Specialty Glass Co., Ltd.) to form a coating film on the base plate. Subsequently, the glass base plate on which the coating film was formed was annealed at 240° C. for 60 seconds. Subsequently, the coated film was subjected to O₂ plasma treatment under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Oka Kogyo Co., Ltd.), and the polymethyl methacrylate portion was selectively dry-etched to obtain a substrate (LS1). In addition, except for using a block copolymer (number average molecular weight 49,000-b-21,000) of polystyrene and polymethyl methacrylate, the same treatment was performed on the smooth surface of one sheet of a 0.8 cm×0.8 cm glass base plate (manufactured by Hiraoka Specialty Glass Co., Ltd.) to obtain a substrate (P1).

(2) 0.2 mL of a 2 wt % propylene glycol monomethyl ether acetate solution of polystyrene (number average molecular weight 18,000) was dropped on a smooth surface of one sheet of a 0.8 cm×0.8 cm glass base plate (manufactured by Hiraoka Specialty Glass Co., Ltd.) and one sheet of a 0.8 cm×0.8 cm polyethylene terephthalate (PET) base plate (manufactured by Mitsubishi Chemical Corporation) to form a coating film on the respective base plates. Subsequently, the coated film was subjected to O₂ plasma treatment under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Oka Kogyo Co., Ltd.) to obtain substrates (Smooth 1 and Smooth 2).

TABLE 1

| | Smooth 1 | LS1 | P1 | Smooth 2 | Smooth 3 | P2 | P3 | P4 | P5 | LS2 | LS3 | LS4 | LS5 | LS6 | LS7 | LS8 |
|---|---|---|---|---|---|---|---|---|---|---|---|---|---|---|---|---|
| Type of | Glass | | | PET | | | | | | | | | | | | |
| base plate | | | | | | | | | | | | | | | | |
| Photosensitive | Polystyrene | | | | Acrylic resin | | | | | | | | | | | |
| resin composition | | | | | (Radical negative resist) | | | | | | | | | | | |
| Pattern | — | Line | Pillar | — | — | Pillar | | | | Line and Space | | | | | | |
| | | and | | | | | | | | | | | | | | |
| | | space | | | | | | | | | | | | | | |
| Width (nm) | 0 | 14 | 20 | 0 | 0 | 100 | 200 | 300 | 500 | 75 | 150 | 200 | 250 | 300 | 500 | 1,000 |

In Table 1, “Smooth” represents a flat substrate used as a control, on which an uneven pattern is not formed. In addition, “Width” represents the width of the projection portion.

### Preparation Example 2

### Preparation of Substrate by Transfer from Argon Fluoride (ArF) Pattern

A radical polymerization negative resist containing a photosensitive resin composition containing an acrylic resin as a main component was used as a photoresist composition. 1 ml of the radical polymerization negative resist was dropped on a 0.8 cm×0.8 cm silicon wafer having a pattern of unevenness (Smooth 3, P2 to P5, and LS2 to LS8) illustrated in Table 1 formed using ArF exposure machine Nikon 5308, the coating film was degassed by placing the coating film under a reduced pressure condition of 100 Pa for 30 minutes, and a radical polymerization negative resist was embedded in the uneven pattern. Subsequently, twelve silicon base plates each having the coating film were exposed in an atmosphere with an exposure amount of 999 J/m² using an ultraviolet irradiation device (HMW-532D, manufactured by ORC Co., Ltd). Subsequently, the film cured by exposure as described above was covered with a base plate prepared by coating a radical polymerization negative resist having a film thickness of 1 μm onto a PET base plate (manufactured by Mitsubishi Chemical Corporation) so that the radical polymerization negative resist having a film thickness of 1 μm was in contact with the film cured, and exposure with an exposure amount of 999 J/m² was repeated five times using an ultraviolet irradiation device (HMW-532D, manufactured by ORC Co., Ltd) in a vacuum, to cure the coating film and the radical polymerization negative resist having a film thickness of 1 μm. After detaching the mold from the cured coating film, the cured coating film was immersed in propylene glycol-1-methyl ether acetate (PGMEA) for 10 minutes and rinsed, and thereafter the nitrogen gas was blown onto the cured coating film to be dried. Subsequently, O₂ plasma treatment was performed on the dried cured coating film under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Ohka Kogyo Co., Ltd.) to obtain a substrate (Smooth 3, P2 to P5 and LS2 to LS8).

### Example 1

### Control of Differentiation of Macrophage

(1) Culture of Macrophage

A cell culture test was performed using the base materials obtained in Production Examples 1 and 2 in a 5% CO₂ environment at a culture temperature of 37° C. Mouse macrophage-derived RAW264 cells (Riken bank RCB0535 was used as Riken bank cells) were used as cells to be cultured. A 10% fetal bovine serum (FBS)-containing RPMI 1640 medium was used as a medium. After the base materials obtained in Production Examples 1 and 2 were placed within wells of a well-attached dish, 2×10⁴ cells were seeded on the surface of each base material. Thereafter, the medium was injected into the wells using a disposable pipette and was cultured for 1 day.

The shapes of the cells after being cultured for 1 day were observed. As a result, it was confirmed that the shapes of the RAW264 cells were changed into an elongated shape in a case where the substrate LS6 (a line-and-space pattern with a width of 300 nm) was used. In general, it is known that the shapes of the cells are round in a case of a resting type macrophage and are elongated in a case of an inflammatory type macrophage. Accordingly, it was suggested that it was possible to induce a trait of a macrophage through culture using the substrate LS6 without using a trait induction compound.

(2) Trait Induction of Macrophage

Next, a trait induction compound was added to a part of the cultured RAW264 cells. Lipopolysaccharide (LPS) and interferon gamma (IFNγ) derived from Escherichia coli were used as compounds for performing trait induction to inflammatory type M1 macrophages. In addition, interleukin 4 (IL-4) was used as a compound for performing trait induction into anti-inflammatory type M2 macrophages. The compounds were added to a medium such that the concentration of each of the compounds in the medium became 10 ng/mL and culturing was performed for 1 day.

(3) Quantitative Determination of Production Amount of Nitrogen Monoxide (NO)

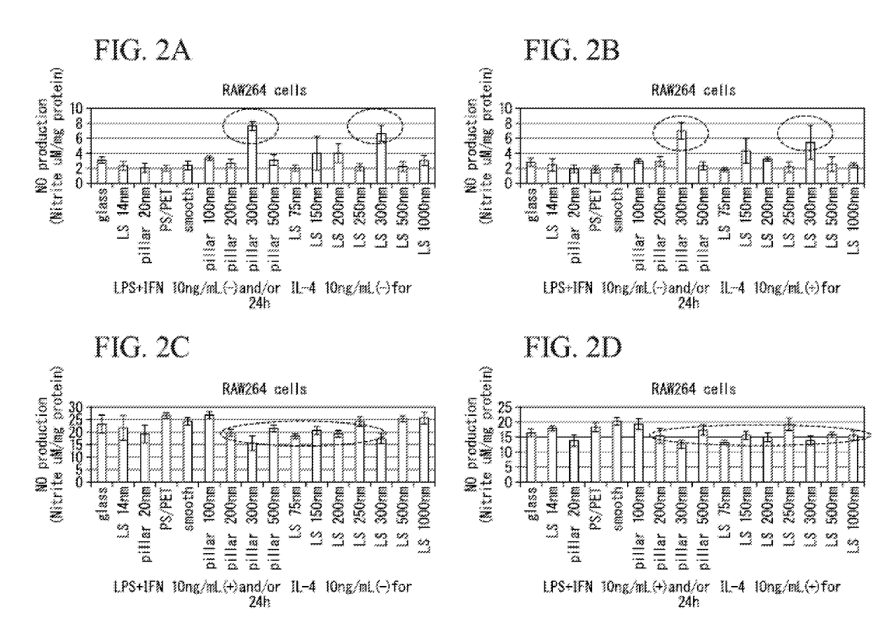

An equal amount of 10% Griess reagent was added to the culture liquid of the RAW264 cells which had been cultured in (2) and the mixture was reacted for 10 minutes at room temperature. In the inflammatory type macrophages, nitrogen monoxide (NO) is synthesized from arginine and the synthesized NO becomes NO₂ after being oxidized. Furthermore, NO₂ is reacted with water to become nitrous acid or nitric acid. In the method in which the Griess reagent is used, it is possible to indirectly evaluate the NO production amount by quantitatively determining the generated nitrous acid. Subsequently, the absorbance at 540 nm was measured using a microplate reader. A calibration curve was previously created using a standard solution of nitrous acid and the concentration of nitrous acid in a culture supernatant was calculated. The results are shown in FIG. 2.

In a case where LPS and IFNγ are added, the NO production amount decreases in some substrates in FIG. 2. In cases where substrates P4 (a pillar pattern with a width of 300 nm) and LS6 (a line-and-space pattern with a width of 300 nm) were used, it was confirmed that the NO production amount increased in the absence of LPS and IFNγ.

From the above, it was confirmed that trait induction into inflammatory type M1 macrophages was achieved using the substrates P4 (a pillar pattern with a width of 300 nm) and LS6 (a line-and-space pattern with a width of 300 nm).

(4) Evaluation of Arginase Activity

(4-1) Enzyme Base Material Reaction Step

The part of the RAW264 cells cultured in (2) was disrupted to prepare a cell-disrupted liquid as an arginase-containing sample. Next, 20 μL of the cell-disrupted liquid was dispensed into a microplate. Subsequently, 20 μL of a Mn solution (5 mM MnCl₂ and 25 mM TrisHCl (pH 7.5)) was dispensed to activate an enzyme. Next, 40 μL of an arginine buffer (0.5 M arginine (pH 9.7)) was dispensed to start an enzyme base material reaction. In addition, a well containing only the cell-disrupted liquid into which a double-concentrated base material buffer was not dispensed was also prepared. Incubation was performed for 2 hours at 37° C. and the enzyme base material reaction was performed.

(4-2) Enzyme Inactivation and Urea Detection Reaction Step

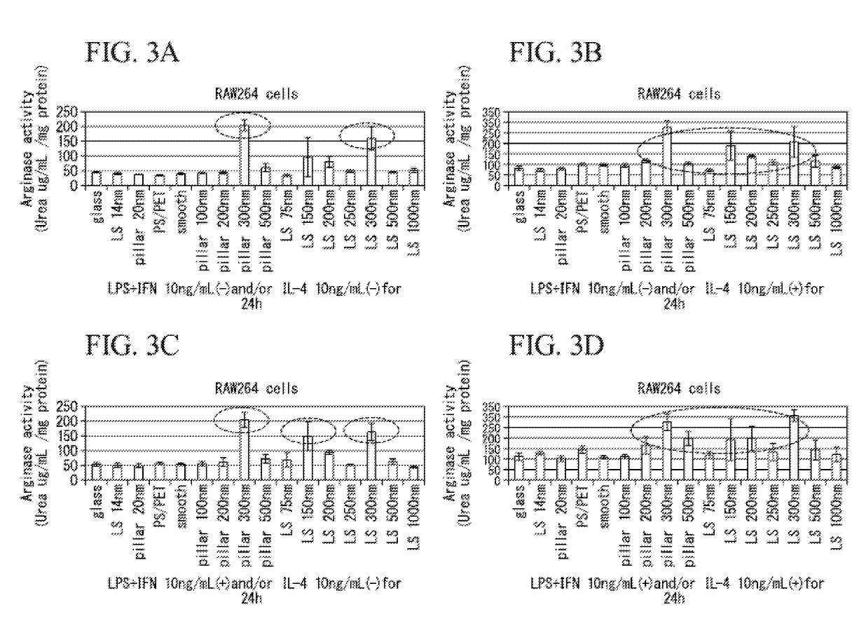

Subsequently, 180 μL of a urea detection reagent (isonitrosopropiophenone 9 wt %/phosphoric acid:concentrated sulfuric acid:water=1:3:7 (volume ratio)) was added to a microplate to stop the enzyme reaction and start a urea detection reaction. Next, incubation was performed for 1 hour and 45 minutes at 95° C. Next, the absorbance at 540 nm of an arginase-containing sample was measured using a microplate reader (Spectra Max i3 manufactured by Molecular Devices) in which a calibration curve had been previously drawn using 0, 5, 10, 20, 40, 80, and 160 μg/mL of urea solutions. The results are shown in FIG. 3.

In cases where the substrates P4 (a pillar pattern with a width of 300 nm), LS3 (a line-and-space pattern with a width of 150 nm), and LS6 (a line-and-space pattern with a width of 300 nm) were used, it was confirmed from FIG. 3 that the arginase activity NO production increased in the absence of IL-4.

From the above, it was confirmed that trait induction into anti-inflammatory type M2 macrophages was achieved using the substrates P4 (a pillar pattern with a width of 300 nm), LS3 (a line-and-space pattern with a width of 150 nm), and LS6 (a line-and-space pattern with a width of 300 nm).

### Example 2

### Differentiation Control of Nerve Cell

(1) Culture of Nerve Precursor Cell

A cell culture test was performed using the base materials obtained in Production Examples 1 and 2 in a 5% CO₂ environment at a culture temperature of 37° C. Rat pheochromocytoma-derived PC12 cells (Riken bank RCB009 was used as Riken bank cells) were used as cells to be cultured. A 10% FBS-containing DMEM high glucose medium was used as a medium. After the base materials obtained in Production Examples 1 and 2 were placed within wells of a well-attached dish, 1×10⁴ cells were seeded on the surface of each base material. Thereafter, the medium was injected into the wells using a disposable pipette and was cultured for 1 day.

The shapes of the cells after being cultured for 1 day were observed. As a result, it was confirmed that neurites appeared from the PC12 cells in cases where the substrates LS3 to LS8 (a line-and-space pattern with a width of 150 nm to 1,000 nm) and P2 to P5 (a pillar pattern with a width of 100 nm to 500 nm) were used.

(2) Differentiation of Nerve Precursor Cell

Next, a differentiation induction compound was added to a part of the cultured PC12 cells. A nerve growth factor (NGF) was used as a differentiation induction substance. A 10% FBS-containing DMEM high glucose medium was used as a medium. The nerve growth factor (NGF) was added to the medium such that the concentration of the nerve growth factor became 50 ng/mL and culturing was performed for 2 days.

(3) Evaluation of Acetylcholine Esterase (AChE) Activity

(3-1) Preparation of Cell-Disrupted Liquid

The part of the PC12 cells cultured in (2) was centrifuged at 4° C. for 10 minutes at 200×g and the supernatant was discarded. Subsequently, the cells were washed with 0.5 mL of a phosphate-buffered saline (PBS) and were centrifuged again at 4° C. for 10 minutes at 200×g. Next, the centrifuged cells were homogenized in 0.5 mL of an assay buffer. Subsequently, the homogenized cells were centrifuged at 4° C. for 10 minutes at 8,000×g. The obtained supernatant was used as a measurement sample to determine acetylcholine esterase activity.

(3-2) Measurement of Acetylcholine Esterase (AChE) Activity

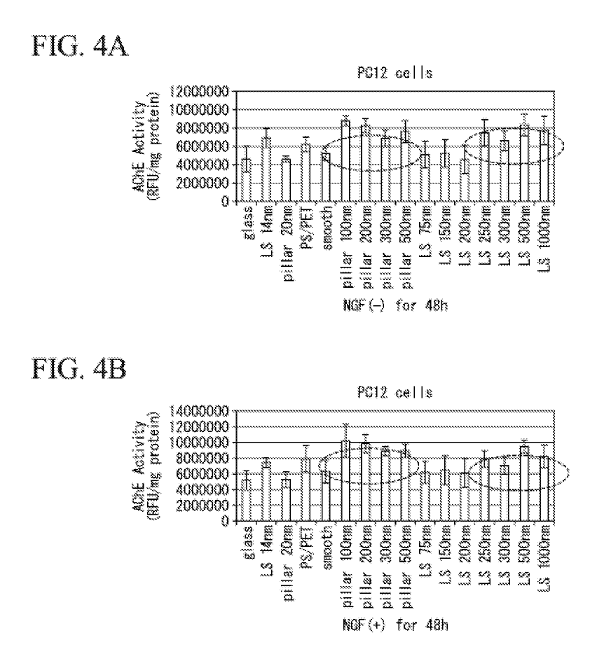

100 μL of the measurement sample obtained in (3-1) was added to a microplate. Subsequently, 50 μL of a 2 mmol/L 5,5′-dithiobis(2-nitrobenzoic acid) (DTNB) solution and 50 μL of 2 mmol/L 1,1-dimethyl-4-acetylthiomethylpiperidinium iodide (MATP+) solution were respectively added to wells and pipetting was performed. A well in which 50 μL of the 2 mmol/L DTNB solution and 50 μL of an assay buffer were developed with respect to 100 μL of the measurement sample was prepared as a control. Next, incubation was performed for 30 minutes at 37° C. Subsequently, the absorbance at 415 nm was measured using a microplate reader. Next, the ACHE activity in the measurement sample was measured from a calibration curve of an AChE standard solution. The results are shown in FIG. 4.

It was confirmed from FIG. 4 that the AChE activity increased in the PC12 cells cultured using P2 to P5 and LS5 to LS8 even in a case where NGF was not added.

(4) Evaluation of Amount of Dopamine

(4-1) Preparation of Cell Culture Supernatant

The culture supernatant of cells cultured using the base materials in (2) was collected.

(4-2) Measurement of Amount of Dopamine

50 μL of the cell culture supernatant obtained in (4-1) was dispensed into wells of a microtiter plate to which a mouse anti-DA antibody of Rat Dopamine (DA) ELISA Kit (manufactured by MyBioSource, Cat. No.: MBS026032) was immobilized.

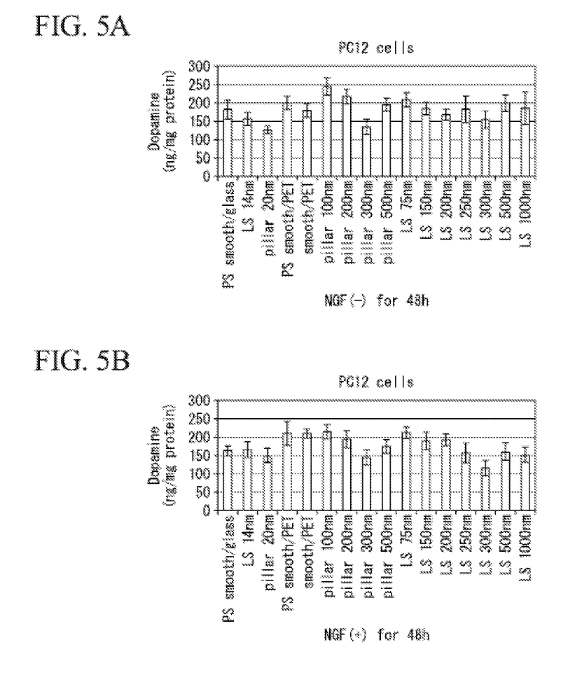

In addition, 50 μL of each standard solution or diluent of the cell culture supernatant was dispensed into the wells. Subsequently, 100 μL of an HRP conjugate reagent was dispensed into each of the wells and incubation was performed for 60 minutes at 37° C. after covering the dispensed reagent. Next, the microtiter plate was washed 4 times using 1×Wash Buffer (washing of 350 μL/well/once). Subsequently, the washing solution was removed, and 50 μL of a chromogenic solution A and 50 μL of a chromogenic solution B were respectively dispensed into the wells. Next, the mixture was gently mixed and incubation was performed for 15 minutes at 37° C. while being shielded from light. Subsequently, 50 μL of a reaction-stopping solution was dispensed to each of the wells, and the absorbance at 450 nm was measured using a microplate reader (Spectra Max i3 manufactured by Molecular Devices). The results are shown in FIG. 5.

It was confirmed from FIG. 5 that the amount of dopamine increased in the PC12 cells cultured using P2, P3, and P5 and LS2 and LS7 even in a case where NGF was not added. In addition, it was confirmed that the amount of dopamine decreased in the PC12 cells cultured using P1 and P4 and LS1 and LS6 even in a case where NGF was not added.

According to the present invention, it is possible to simply and efficiently induce undifferentiated cells to have a constant trait.

The present invention can be used for improving biocompatibility through surface processing of a medical material.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

1 . . . linear shape convex portion, 2 . . . dot shape convex portion, 10, 20 . . . base material

What is claimed is:

1. A trait induction method of undifferentiated cells, comprising:
culturing undifferentiated cells on a base material which has an uneven pattern on the surface to which the cells adhere and of which the width of the unevenness is 1 nm to 1,000 nm.

2. The trait induction method of undifferentiated cells according to claim 1,
wherein the undifferentiated cells are stem cells or precursor cells.

3. The trait induction method of undifferentiated cells according to claim 1,
wherein the uneven pattern is a lattice shape, a radial shape, a shape in which a polygon is continuous on a plane, a labyrinth shape, a linear shape, or a dot shape.

4. The trait induction method of undifferentiated cells according to claim 1,
wherein the culture period of time of the undifferentiated cells is 1 hour to 100 hours.
